(19) United States
(12) Plant Patent Application Publication
(10) Pub. No.: US 2013/0326766 P1
(43) Pub. Date: Dec. 5, 2013

(54) ROSA HYBRID VARIETY DENOMINATION 'POULPAL039'

(50) ;

(71) Applicant: Mogens Nyegaard Olesen, Fredensborg (DK)

(72) Inventor: Mogens Nyegaard Olesen, Fredensborg (DK)

(21) Appl. No.: 13/507,018

(22) Filed: May 31, 2012

(51) Int. Cl. A01H 5/00

(52) U.S. Cl. PLT/150

(57) ABSTRACT

A new garden rose plant of the Compact Floribunda class which has abundant, red flowers and attractive foliage. This new and distinct variety has shown to be uniform and stable in the resulting generations from asexual propagation.

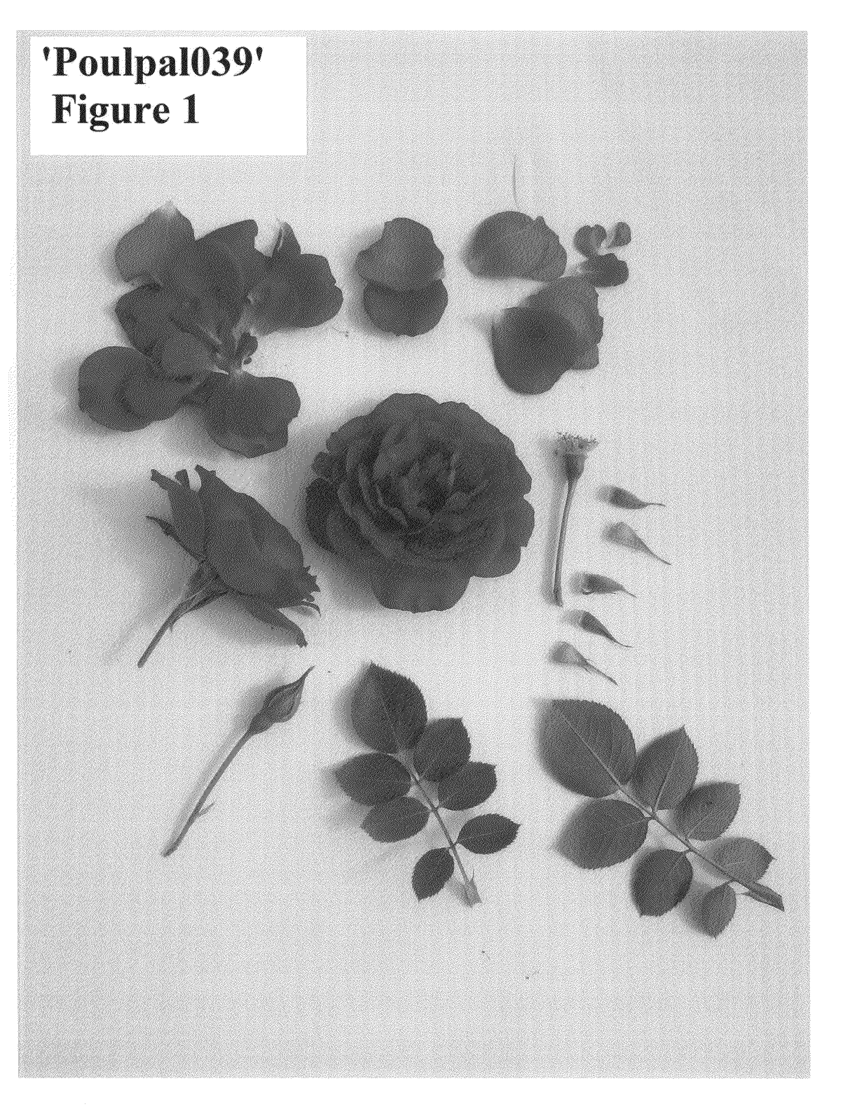

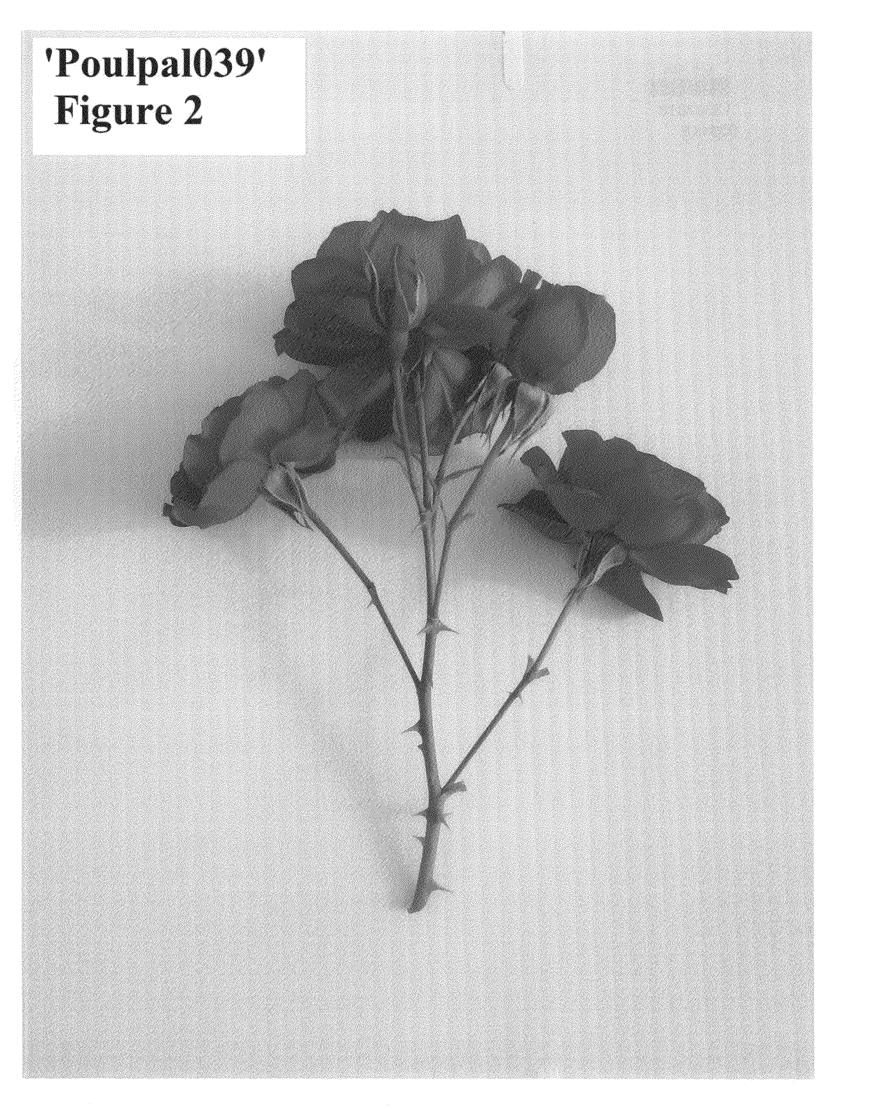

### BOTANICAL DESIGNATION

Rosa hybrid

### VARIETY DENOMINATION

‘Poulpal039’

### SUMMARY OF THE INVENTION

The present invention constitutes a new and distinct variety of garden rose plant which originated from a controlled crossing between the female seed parent, an unnamed seedling, and the male pollen parent, also an unnamed seedling.

The two parents were crossed during the summer of 1998 and the resulting seeds were planted in a controlled environment in Fredensborg, Denmark. The new variety, named ‘Poulpal039’, originated as a single seedling from the stated cross.

The new variety may be distinguished from its male pollen parent and female seed parent primarily by flower coloration and growth habit.

The objective of the hybridization of this rose variety was to create a new and distinct variety for garden use with unique qualities, such as:
1. Uniform and abundant red flowers;
2. Vigorous, but compact growth when propagated both as a budded rose and on its own roots;
3. Exceptional disease resistance.

This combination of qualities is not present in previously available commercial cultivars of this type, known to the inventor, and distinguish ‘Poulpal039’ from all other varieties of which we are aware.

As part of the rose development program, Mogens N. Olesen germinated the seeds from the aforementioned hybridization during winter of 1998 and conducted evaluations on the resulting seedlings in a controlled environment in Fredensborg, Denmark. ‘Poulpal039’ was selected in the spring of 1999 by the inventor as a single plant from the progeny of the aforementioned hybridization.

Asexual reproduction of ‘Poulpal039’ by traditional budding and rooted cuttings was first done by Mogens N. Olesen in the nursery in Fredensborg, Denmark in July, 1999. This initial and other subsequent asexual propagations conducted in controlled environments have demonstrated that the characteristics of ‘Poulpal039’ are true to type and are transmitted from one generation to the next.

### DESCRIPTION OF THE DRAWING

The accompanying color illustrations show as true as is reasonably possible to obtain in color photographs of this type, the typical characteristics of the buds, flowers, leaves, and stems, of ‘Poulpal039’. Specifically illustrated in the drawing are flowers at various stages of development, flower in parts, leaves, and stems.

### DETAILED DESCRIPTION OF THE VARIETY

The following is a description of ‘Poulpal039’, as observed in its growth in in a field nursery in Bakersfield, Calif. 2 years of age, and were grown on their own roots. Color references are made using The Royal Horticultural Society (London, England) Colour Chart, 2001, except where common terms of color are used.

For a comparison, several physical characteristics of the rose variety ‘Poulchris’, U.S. Plant Pat. No. 11,151 are compared to ‘Poulpal039’ in Chart 1.

CHART 1

| | ‘Poulpa1039’ | ‘Poulchris’ |
|---|---|---|
| Petal Count | 50 | 55 to 65 |
| Flower Diameter | 70-80 mm | 60 mm |
| Petal color after | Red Group 46B | Red Group 53A |
| opening, upper | | |
| surface | | |

Flower and flower bud:
   Blooming habit.—Continuous.
   Flower bud.—Size: Upon opening, 30 mm in length from base of receptacle to end of bud. Bud diameter is 12 mm. Bud form: Ovoid. Bud color: As sepals divide petals are Red Group 53B. Sepal inner surface: Color: Yellow-Green Group 144C. Surface: Smooth. Sepal outer surface: Color: Yellow-Green Group 144A with intonations of Greyed-Orange Group 174A. Texture: Smooth. Sepal Shape: Apex: Cirrhose. Base: Flat at union with receptacle. Sepal Margin: Margins have weak foliaceous appendages on three of the five sepals. Sepal size: 25 mm long by 7 mm wide. Receptacle: Texture: Smooth. Size: 9 mm in height by 5 mm wide. Color: Yellow-Green Group 144A. Anthocyanic pigments the color of Greyed Group observed. Shape: Elliptical. Pedicel: Surface: Smooth. Length: 25 to 40 mm. Diameter: 2 mm on average. Color: Yellow-Green Group 144A. Strength: Moderate. Peduncle: Length: 30 to 35 cm. Diameter: 3 mm. Color: Yellow-Green Group 145A.
   Flower bud development.—Flower buds are borne in clusters of 3 to 7 flower buds per stem, resembling a corymb.
   Flower bloom.—Fragrance: Moderate old rose scent. Duration: The blooms have a duration on the plant of approximately 14 days. Petals fall cleanly away from plant after flowers have fully matured. Size: Flower diameter is 70-80 mm when open. Flower depth is 25 mm. Flower shape: General shape is a rosette, very double flower, with many slightly overlapping petals of different sizes. Shape of flower, side view: The upper portion is flat, and the lower portion is concave.
   Petalage.—Under normal conditions, flowers have 50 petals total, 5 to 7 of which are petaloids.
   General tonality of flower.—Open flowers are Red Group 45B and remain this color as the flower matures.
   Petal color.—Upon opening, inner and outer petals: Upper surface: Red Group 46A to 46B. Lower surface: Red Group 53C. Basal petal spots: Yellow Group 7B on the upper and lower surface. After opening, outer and inner petals: Upper surface: Red Group 46B. Lower surface: Red Group 53C. Basal petal spots, after opening: Upper surface: Yellow Group 5D. Lower surface: Yellow Group 5D.
   Petals.—Petal Reflex: Weak. Margin: Entire and uniform. No undulations of margin observed. Shape: Narrow elliptic. Apex shape: Rounded, occasionally cuspidate. Base shape: Acute. Size: 40 mm (l)×35 mm (w). Texture: Smooth. Thickness: Average.
   Petaloids.—Size: 15 mm (l) by 10 mm (w). Quantity: 5 to 7. Shape: Irregular. Apex is rounded and the base is acute. Color: Upper surface is Red Group 46B. The lower surface is Red Group 53C. Basal petal spots are Yellow Group 5D.
   Reproductive organs.—Pollen: None observed. Anthers: Size: 1 mm in length. Color: Greyed-Orange Group N167B. Quantity: 45 on average. Filaments: Color: Yellow Group 13C. Length: 6 mm. Pistils: Length: 5 mm. Quantity: 35 on average. Stigmas: Color: Greyed-Yellow Group 162B. Styles: Color: Red-Purple Group 58B. Location of stigmas: Inferior in location relative to the length of the filaments and the height of the anthers. Hips: None Observed.
Plant:
   Plant growth.—Upright, bushy. Plants are 50 cm in height, and 50 cm wide.
   Stems.—Color: Juvenile growth: Yellow-Green Group 144B. Mature growth: Yellow-Green Group 144B. Length: On average, canes are 25 cm from the base of the plant to the flowering portion. Diameter: 5 to 6 mm. Internodes: On mature canes, there is an average distance of 35 mm between nodes. Surface Texture: Young wood: Smooth. Older wood: Smooth.
   Long prickles.—Incidence: 1 to 3 prickles per 10 cm of stem. Size: Average length of prickles on mature stems is 9 mm. Shape: Upper portion is linear. Lower portion is concave. Color: Mature and juvenile prickles are Greyed-Yellow Group 160C with Greyed-Red Group 180D.
   Plant foliage.—Compound leaf: 110 mm (l)×70 (w). Quantity: 3 leaves per 10 cm of stem on average. Leaf bearing angle to the stem: 90 degrees. Color of juvenile foliage: Upper side: Yellow-Green Group 146B. Lower side: Yellow-Green Group 146C. Color of mature foliage: Upper side: Yellow-Green Group 147B. Lower side: Yellow-Green Group 146C.
   Plant leaves and leaflets.—Stipules: Size: 15 mm in length. Quantity: 2 per compound leaf. Shape: Linear, slightly broad based with outward extending apecies. Margins: Finely serrated with few stipitate glands. Color: Yellow-Green Group 144A. Petiole: Length: 20 mm. Diameter: 2 mm. Upper surface: Color: Yellow-Green Group 144A. Lower surface: Color: Yellow-Green Group 144B. Rachis: Length: 50 mm. Upper surface: Color: Yellow-Green Group 144A. Lower surface: Color: Yellow-Green Group 144B. Leaflet: Quantity: Normal number of leaflets leaves in middle of the stem is 7 leaflets. Margins: Serrated. Size: Average size of the terminal leaflet on normal leaves is 40 mm in length by 30 mm wide. Shape: Generally elliptical. Base: Rounded. Apex: Acute. Cuspidate. Texture: Smooth. Thickness: Average. Arrangement: Odd pinnate. Venation: Reticulate. Glossiness: Not glossy.
   Disease resistance.—Above average resistance to powdery and downy mildew, rust, black spot, and Botrytis under normal growing conditions.
   Cold hardiness.—The variety is tolerant to USDA Cold Hardiness Zone 6.
   Heat tolerance.—The variety has been found to be suitable for climate conditions found in the American Horticulture Society heat zone 7.

1. A new and distinct variety of rose plant of the Compact Floribunda rose class named ‘Poulpal039’, substantially as illustrated and described herein, due to its abundant red flowers, disease resistance, and extended period of bloom.
